SEQUENCE LISTING (68 sequences)

SEQ ID NO: 1 - DNA, 21 bp, Artificial Sequence
  Description of Artificial Sequence Synthetic probe
atagcctagtatcacatacac

SEQ ID NO: 2 - DNA, 21 bp, Artificial Sequence
  Description of Artificial Sequence Synthetic probe
atagcctaggttcacatacac

SEQ ID NO: 3 - DNA, 21 bp, Artificial Sequence
  Description of Artificial Sequence Synthetic probe
attggccatattcctacttgc

SEQ ID NO: 4 - DNA, 21 bp, Artificial Sequence
  Description of Artificial Sequence Synthetic probe
tattattgcatgcactgggtg

SEQ ID NO: 5 - DNA, 21 bp, Artificial Sequence
  Description of Artificial Sequence Synthetic probe
cagtcgtgttcttcagattca

SEQ ID NO: 6 - DNA, 21 bp, Artificial Sequence
  Description of Artificial Sequence Synthetic probe
tggctcctctcaaaagagtta

SEQ ID NO: 7 - DNA, 21 bp, Artificial Sequence
  Description of Artificial Sequence Synthetic probe
agttcgtacaagttcgtggat

SEQ ID NO: 8 - DNA, 21 bp, Artificial Sequence
  Description of Artificial Sequence Synthetic probe
ggataaagatgctaggaatgt

SEQ ID NO: 9 - DNA, 21 bp, Artificial Sequence
  Description of Artificial Sequence Synthetic probe
ttgtccggtagataaaggcag

SEQ ID NO: 10 - DNA, 21 bp, Artificial Sequence
  Description of Artificial Sequence Synthetic probe
tcagttttgcccggtggataa

SEQ ID NO: 11 - DNA, 21 bp, Artificial Sequence
  Description of Artificial Sequence Synthetic probe
acttcggtcgattacttttac

SEQ ID NO: 12 - DNA, 21 bp, Artificial Sequence
  Description of Artificial Sequence Synthetic probe
aacgtgctgcgctactaggtg

SEQ ID NO: 13 - DNA, 21 bp, Artificial Sequence
  Description of Artificial Sequence Synthetic probe
tcgctggtgttgacttgagaa

SEQ ID NO: 14 - DNA, 21 bp, Artificial Sequence
  Description of Artificial Sequence Synthetic probe
cagtgacatgtgggcgttata

SEQ ID NO: 15 - DNA, 21 bp, Artificial Sequence
  Description of Artificial Sequence Synthetic probe
gtttcactggttccattgtgt

SEQ ID NO: 16 - DNA, 21 bp, Artificial Sequence
  Description of Artificial Sequence Synthetic probe
ctgggtccatggtgttgaagc

SEQ ID NO: 17 - DNA, 21 bp, Artificial Sequence
  Description of Artificial Sequence Synthetic probe
tacttccttggaacgggtcaa

SEQ ID NO: 18 - DNA, 21 bp, Artificial Sequence
  Description of Artificial Sequence Synthetic probe
gagcagcgagcgacttggcag

SEQ ID NO: 19 - DNA, 21 bp, Artificial Sequence
  Description of Artificial Sequence Synthetic probe
actgcagctcacctttatggc

SEQ ID NO: 20 - DNA, 21 bp, Artificial Sequence
  Description of Artificial Sequence Synthetic probe
cttaacacgataaccggtgct

SEQ ID NO: 21 - DNA, 21 bp, Artificial Sequence
  Description of Artificial Sequence Synthetic probe
ttatagcctgttatcacatac

SEQ ID NO: 22 - DNA, 21 bp, Artificial Sequence
  Description of Artificial Sequence Synthetic probe
aggtagtttcaatgtagcttc

SEQ ID NO: 23 - DNA, 23 bp, Artificial Sequence
  Description of Artificial Sequence Synthetic probe
catgaatcatgtttattggactc

SEQ ID NO: 24 - DNA, 21 bp, Artificial Sequence
  Description of Artificial Sequence Synthetic probe
ctgatcgctcgccttaaaaga

SEQ ID NO: 25 - DNA, 21 bp, Artificial Sequence
  Description of Artificial Sequence Synthetic probe
tggatttgagtgatggcagtt

SEQ ID NO: 26 - DNA, 21 bp, Artificial Sequence
  Description of Artificial Sequence Synthetic probe
tttttacttcgatctcaaatc

SEQ ID NO: 27 - DNA, 21 bp, Artificial Sequence
  Description of Artificial Sequence Synthetic probe
cactctgtgtcgattttacaa

SEQ ID NO: 28 - DNA, 22 bp, Artificial Sequence
  Description of Artificial Sequence Synthetic probe
agttcatgctctaagtcggttc

SEQ ID NO: 29 - DNA, 21 bp, Artificial Sequence
  Description of Artificial Sequence Synthetic probe
cctattgtcgtatacactgga

SEQ ID NO: 30 - DNA, 22 bp, Artificial Sequence
  Description of Artificial Sequence Synthetic probe
cggacaattcttgaactctggt

SEQ ID NO: 31 - DNA, 21 bp, Artificial Sequence
  Description of Artificial Sequence Synthetic probe
tgtgagtctatcgggcgcttg

SEQ ID NO: 32 - DNA, 21 bp, Artificial Sequence
  Description of Artificial Sequence Synthetic probe
tgtcctctggagtaataagtt

SEQ ID NO: 33 - DNA, 23 bp, Artificial Sequence
  Description of Artificial Sequence Synthetic probe
tgaacgtctagttattataacaa

SEQ ID NO: 34 - DNA, 21 bp, Artificial Sequence
  Description of Artificial Sequence Synthetic probe
ataaaggtaataggaaggtgg

SEQ ID NO: 35 - DNA, 21 bp, Artificial Sequence
  Description of Artificial Sequence Synthetic probe
cagtcgtgtttattggattca

SEQ ID NO: 36 - DNA, 20 bp, Artificial Sequence
  Description of Artificial Sequence Synthetic probe
tgaacgtcattagatcataa

SEQ ID NO: 37 - DNA, 21 bp, Artificial Sequence
  Description of Artificial Sequence Synthetic probe
ggtttaagattgtattgactg

SEQ ID NO: 38 - DNA, 21 bp, Artificial Sequence
  Description of Artificial Sequence Synthetic probe
aacgtacaagtccggacatga

SEQ ID NO: 39 - DNA, 22 bp, Artificial Sequence
  Description of Artificial Sequence Synthetic probe
acggtagtttggagtgaagtgc

SEQ ID NO: 40 - DNA, 21 bp, Artificial Sequence
  Description of Artificial Sequence Synthetic probe
gagcagcgagcgacttggcag

SEQ ID NO: 41 - DNA, 21 bp, Artificial Sequence
  Description of Artificial Sequence Synthetic probe
ttctcgactgatggccttggt

SEQ ID NO: 42 - DNA, 21 bp, Artificial Sequence
  Description of Artificial Sequence Synthetic probe
gggtcctaatagatgccatgt

SEQ ID NO: 43 - DNA, 21 bp, Artificial Sequence
  Description of Artificial Sequence Synthetic probe
cacagaggtgcaagaggtggg

SEQ ID NO: 44 - DNA, 21 bp, Artificial Sequence
  Description of Artificial Sequence Synthetic probe
gcttcggagacttgggtttgc

SEQ ID NO: 45 - DNA, 21 bp, Artificial Sequence
  Description of Artificial Sequence Synthetic probe
actgttctaccacttgacgca

SEQ ID NO: 46 - DNA, 21 bp, Artificial Sequence
  Description of Artificial Sequence Synthetic probe
ttatcacatgcactgggggag

SEQ ID NO: 47 - DNA, 21 bp, Artificial Sequence
  Description of Artificial Sequence Synthetic probe
gcatgagtgacggcagaggtg

SEQ ID NO: 48 - DNA, 21 bp, Artificial Sequence
  Description of Artificial Sequence Synthetic probe
gtgcacaagaaacatactaac

SEQ ID NO: 49 - DNA, 21 bp, Artificial Sequence
  Description of Artificial Sequence Synthetic probe
atagcctattatcacatacac

SEQ ID NO: 50 - DNA, 21 bp, Artificial Sequence
  Description of Artificial Sequence Synthetic probe
atagcctattgtcacatacac

SEQ ID NO: 51 - DNA, 21 bp, Artificial Sequence
  Description of Artificial Sequence Synthetic probe
atagcctattattgcatacac

SEQ ID NO: 52 - DNA, 24 bp, Artificial Sequence
  Description of Artificial Sequence Synthetic probe
cagtaggaatgtggcttcttcgga

SEQ ID NO: 53 - DNA, 24 bp, Artificial Sequence
  Description of Artificial Sequence Synthetic probe
tattaggaatgtggcttcttcgga

SEQ ID NO: 54 - DNA, 24 bp, Artificial Sequence
  Description of Artificial Sequence Synthetic probe
cagtaggaatgtagcttctccgga

SEQ ID NO: 55 - DNA, 24 bp, Artificial Sequence
  Description of Artificial Sequence Synthetic probe
cattaggaatgtggctccctcggg

SEQ ID NO: 56 - DNA, 21 bp, Artificial Sequence
  Description of Artificial Sequence Synthetic probe
gtttcactgggtccattgtgt

SEQ ID NO: 57 - DNA, 21 bp, Artificial Sequence
  Description of Artificial Sequence Synthetic probe
gtttcactgggtccatggtgt

SEQ ID NO: 58 - DNA, 21 bp, Artificial Sequence
  Description of Artificial Sequence Synthetic probe
atgaatcatgtttattggact

SEQ ID NO: 59 - DNA, 24 bp, Artificial Sequence
  Description of Artificial Sequence Synthetic primer
gcatatcaataagcggaggaaaag

SEQ ID NO: 60 - DNA, 18 bp, Artificial Sequence
  Description of Artificial Sequence Synthetic primer
ggtccgtgtttcaagacg

SEQ ID NO: 61 - DNA, 18 bp, Artificial Sequence
  Description of Artificial Sequence Synthetic primer
tccgtaggtgaacctgcg

SEQ ID NO: 62 - DNA, 20 bp, Artificial Sequence
  Description of Artificial Sequence Synthetic primer
tcctccgcttattgatatgc

SEQ ID NO: 63 - DNA, 20 bp, Artificial Sequence
  Description of Artificial Sequence Synthetic primer
agcttgacttcgcagatcgg

SEQ ID NO: 64 - DNA, 21 bp, Artificial Sequence
  Description of Artificial Sequence Synthetic primer
ctgaacgcctctaagtcagaa

SEQ ID NO: 65 - DNA, 20 bp, Artificial Sequence
  Description of Artificial Sequence Synthetic primer
ttaccacagggataactggc

SEQ ID NO: 66 - DNA, 21 bp, Artificial Sequence
  Description of Artificial Sequence Synthetic primer
cagacgacttgaatgggaacg

SEQ ID NO: 67 - DNA, 24 bp, Artificial Sequence
  Description of Artificial Sequence Synthetic probe
atgaacgtctagttattataacaa

SEQ ID NO: 68 - DNA, 22 bp, Artificial Sequence
  Description of Artificial Sequence Synthetic probe
agatcataacataaaaaaactt

(19) United States
(12) Patent Application Publication
(10) Pub. No.: US 2006/0216723 A1
(43) Pub. Date: Sep. 28, 2006

(54) High through-put detection of pathogenic yeasts in the genus trichosporon

(75) Inventors: Mara R. Diaz, Key Biscayne, FL (US); Jack Fell, Miami, FL (US)

Correspondence Address: VENABLE LLP, P.O. BOX 34385, WASHINGTON, DC, 20045-9998, US

(73) Assignee: University of Miami, Miami, FL (US)

(21) Appl. No.: 11/134,619

(22) Filed: May 23, 2005

Related U.S. Application Data
(60) Provisional application No. 60/572,975, filed on May 21, 2004.

(51) Int. Cl. C12Q 1/68; C07H 21/04

(52) U.S. Cl. 435/6; 536/24.1

(57) ABSTRACT

The emergence of opportunistic and antifungal resistant strains has given rise to an urgent need for a rapid and accurate method for the detection of fungal pathogens. In this application, we demonstrate the detection of medically important fungal pathogens at the species level. The present method, which is based on a nucleotide hybridization assay, consists of a combination of different sets of fluorescent beads covalently bound to species specific capture probes. Upon hybridization, the beads bearing the target amplicons are classified by their spectral addresses with a 635 nm laser. Quantitation of the hybridized biotinylated amplicon is based on the fluorescent detection with a 532 nm laser. Using this technology we designed and tested various multiplex formats, the performance of forty eight species specific and group specific capture probes designed from sequence analysis in the D1/D2 region of ribosomal DNA, internal transcribed spacer regions (ITS), and intergenic spacer region (IGS). Species-specific biotinylated amplicons (>600 bp) were generated with three sets of primers to yield fragments from the three regions. The developed assay was specific and relatively fast, as it discriminated species differing by one nucleotide and required less than 50 min following amplification to process a 96 well plate with the capability to detect up to 100 species per well. The sensitivity of the assay allowed the detection as low as 10² genome molecules in PCR reactions and 10⁷ to 10⁸ molecules of biotinylated amplification product. This technology provided a rapid means of detection of Trichosporon species and had the flexibility to identify species in a multiplex format by combining different sets of beads. The assay can be expanded to include all known pathogenic fungal species.

This research was supported by NIH grant 1-UO1 AI53879-01. The United States Government has certain rights in the invention.

### BACKGROUND OF THE INVENTION

1. Field of the Invention

The invention relates to species-specific nucleic acid probes and a method for using the probes to detect fungal infection.

2. Background Information

The advances of medical technologies and treatments e.g., chemotherapy, organ transplantation, antimicrobial therapies, have contributed to the dissemination of fungal infections. For example, the incidence of invasive fungal infestation among organ transplant recipients has been reported as high as 59% (16). Among fungal diseases, deep-seated trichosporonosis is one of the leading causes of mortality in immunocompromised patients (37). The disease is associated with severe conditions that cause morbidity such as respiratory and renal failure, intravascular coagulation syndrome and immunocompromised patients in neutropenic state (24). The causative agents of the disease includes: Trichosporon inkin, T. ovoides, T. cutaneum, T. asahii, T. asteroides and T. mucoides. The clinical cases caused by opportunistic fungal infection are constantly rising and new species within the genus are emerging as new opportunistic pathogens (15, 26, 35). For example, two recent clinical cases have confirmed the emergence of Trichosporon loubieri as a new human pathogen that can cause death if the disease is left unattended (26). To make matters worse, the prognosis for patients is relatively poor. In view of the severity of the situation, a rapid and correct identification method is important for efficient and prompt therapy. However, most clinical laboratories rely on methods that employ phenotypic characteristics that can be time consuming and not very accurate.

There is a need for a prompt, accurate and reliable identification of yeast pathogens. To date, many common fungal species are not detected by common serological and microscopic tests (9, 30). Most of the conventional fungal diagnostic kits, such as the API® kit (bioMerieux Vitek, Hazelwood, Mo.) and ID 32C (bioMerieux, Marcy l'Etoile, France) allow identification based on physiological and biochemical characteristics, which sometimes can be laborious, inconclusive and do not provide accurate resolution at species level (10). During the last decade, several molecular techniques have been employed for the detection of fungal pathogens using gene sequence analyses combined with species specific primers or hybridization probes designed in 18S rDNA (20), 26SrDNA (7, 14), mitochondrial DNA (12) and ITS region (1, 10, 33). Some of the PCR based methods have been employed for the detection of clinically relevant Trichosporon species (10, 24, 32). This important genus is normally found in soil and fresh water and has been known to cause white piedra, hypersensitive pneumonia and deep-seated infections (38). A nested PCR was developed for two of the most common species eg. T. asahii and T. mucoides, both species cause deep-seated infections (24). Similarly, Sugita et al. (32), described a PCR analysis that employed one set of genus specific primers to detect all Trichosporon species. Most recently, a multiplex PCR based method in conjunction with microchip electrophoresis (PCR-ME) was developed for the identification of several species of Trichosporon and Candida. However, PCR-ME technology, which is based on length variability of PCR products, can be of little value for species displaying similar length PCR products. Also, as observed with any gel electrophoresis identification method, non-specific bands can translate into ambiguous results. Even though some of the PCR approach methods are fairly fast, these analyses focus on a limited number of Trichosporon species and do not provide the resolution necessary to differentiate among closely related species.

### SUMMARY OF THE INVENTION

With rapid advances in molecular biology, combined with our in-house fungal database and the public accessibility of microbial sequence data, we developed a rapid and simple assay with the high-throughput capability to identify all the species within the genus Trichosporon. In a preferred embodiment, this method can be used with a novel technology based upon to the principles of flow cytometry, the Luminex® 100™. This Technology uses polystyrene beads (microspheres) that are internally dyed with two spectrally distinct fluorescent dyes. Using precise concentrations of these fluorescent dyes, an array consisting of 100 distinct sets of color-coded microspheres is produced. Each microsphere set can carry a different reactant on its surface. Since individual beads can be distinguished by their spectral address, once the sets are combined, up to 100 different analytes can be measured simultaneously in a single reaction vessel. Each such bead within the set is said to have a specific spectral address.

The polystyrene microspheres are coated with carboxyl groups, which bind covalently to species-specific nucleic acid probes by the carbodiimide coupling method—EDC (11). A captured probe microsphere-based hybridization assay uses PCR biotinylated amplicon target DNA that is inoculated into the microsphere bead mixture containing species-specific probes of interest. By adding a reporter molecule (streptavidin R-phycoerythrin) all hybridized species-specific amplicons captured by their complementary nucleotide sequence in the microsphere beads are recognized by the fluorescence of the reporter molecule. The median fluorescent intensity (MFI) of the reporter molecule is then used to quantify the amount of DNA bound to the beads.

This technology has been adapted to a wide variety of applications involving human single nucleotide polymorphisms (SNPs) (39), bacterial identification (6, 29, 40), Y chromosome SNPs analysis (4), and kinase assays for drug discovery (25).

We present a sensitive molecular method, which is rapid and simple to perform, rendering the assay a practical method for clinical use. This technology, which was adapted to identify the species within the genus Trichosporon can be expanded to include other pathogenic fungal species. To our knowledge, this is the first application of Luminex xMap® technology for the detection of fungal pathogens.

Accordingly, it is one object of the invention to provide capture probes useful for the detection of fungal infections, in particular for the identification of species within the genus Trichosporon. The capture probes of the invention will generally comprise oligonucleotides of 15-25 bases in length, preferably 20-22 bases, but may be larger or smaller. Oligonucleotides of 16, 17 and 18 bases in length are also considered to be particularly useful. Examples of preferred capture probes of the invention are presented in Table 2.

TABLE 2
List of probe sequences used for Trichosporon species specific and group specific identification

| | | | | |
|---|---|---|---|---|
| | | | | Concentration |
| Probe | rDNA region | Specificity | Probe Sequence (5′-3′) | (nmol) |

| | | | | | |
|---|---|---|---|---|---|
| P1 | D1/D2 | T. brassicae | ATAGCCTAGTATCACATACAC | (SEQ ID NO:1) | 0.1 |
| P2 | D1/D2 | T. montevideense/T. domesticum | ATAGCCTAGGTTCACATACAC | (SEQ ID NO:2) | 0.1 |
| P3c | IGS | T. scarabeorum | ATTGGCCATATTCCTACTTGC | (SEQ ID NO:3) | 0.4 |
| P4 | D1/D2 | T. monoliiforme | TATTATTGCATGCACTGGGTG | (SEQ ID NO:4) | 0.1 |
| P5 | D1/D2 | T. jirovecil/T. cutaneum | CAGTCGTGTTCTTCAGATTCA | (SEQ ID NO:5) | 0.1 |
| P6 | ITS | T. laibachii/T. multisporum | TGGCTCCTCTCAAAAGAGTTA | (SEQ ID NO:6) | 0.1 |
| P7b | IGS | T. multisporum | AGTTCGTACAAGTTCGTGGAT | (SEQ ID NO:7) | 0.2 |
| P8 | D1/D2 | T. gracile | GGATAAAGATGCTAGGAATGT | (SEQ ID NO:8) | 0.1 |
| P9 | D1/D2 | T. veenhuisii | TTGTCCGGTAGATAAAGGCAG | (SEQ ID NO:9) | 0.1 |
| P10 | D1/D2 | T. loubieri | TCAGTTTTGCCCGGTGGATAA | (SEQ ID NO:10) | 0.2 |
| P11 | ITS | T. mucoides | ACTTCGGTCGATTACTTTTAC | (SEQ ID NO:11) | 0.2 |
| P11b | IGS | T. mucoides | AACGTGCTGCGCTACTAGGTG | (SEQ ID NO:12) | 0.4 |
| P12 | ITS | T. cutaneum | TCGCTGGTGTTGACTTGAGAA | (SEQ ID NO:13) | 0.1 |
| P12b | IGS | T. cutaneum | CAGTGACATGTGGGCGTTATA | (SEQ ID NO:14) | 0.2 |
| P13 | ITS | T. ovoides | GTTTCACTGGTTCCATTGTGT | (SEQ ID NO:15) | 0.1 |
| P14 | ITS | T. inkin | CTGGGTCCATGGTGTTGAAGC | (SEQ ID NO:16) | 0.1 |
| P15 | D1/D2 | T. asteroids/ | TACTTCCTTGGAACGGGTCAA | (SEQ ID NO:17) | 0.1 |
| | | T. japonicum/T. asahii | | | |
| P16b | IGS | T. japonicum | GAGCAGCGAGCGACTTGGCAG | (SEQ ID NO:18) | 0.2 |
| P17 | D1/D2 | T. faecale | ACTGCAGCTCACCTTTATGGC | (SEQ ID NO:19) | 0.2 |
| P18b | IGS | T. aquatile | CTTAACACGATAACCGGTGCT | (SEQ ID NO:20) | 0.2 |
| P19 | D1/D2 | T. caseorum | TTATAGCCTGTTATCACATAC | (SEQ ID NO:21) | 0.4 |
| P20 | D1/D2 | T. guehoe | AGGTAGTTTCAATGTAGCTTC | (SEQ ID NO:22) | 0.2 |
| P21b | D1/D2 | T. porosum | CATGAATCATGTTTATTGGACTC | (SEQ ID NO:23) | 0.1 |
| P22 | ITS | Ovoides cluster | CTGATCGCTCGCCTTAAAAGA | (SEQ ID NO:24) | 0.1 |
| P23b | ITS | T. smithiae | TGGATTTGAGTGATGGCAGTT | (SEQ ID NO:25) | 0.2 |
| P24 | ITS | T. debeurmannianum | TTTTTACTTCGATCTCAAATC | (SEQ ID NO:26) | 0.1 |
| P25b | ITS | T. sporotrichoides | CACTCTGTGTCGATTTTACAA | (SEQ ID NO:27) | 0.2 |
| P25c | IGS | T. sporotrichoides | AGTTCATGCTCTAAGTCGGTTC | (SEQ ID NO:28) | 0.2 |
| P26 | D1/D2 | T. dehoogii | CCTATTGTCGTATACACTGGA | (SEQ ID NO:29) | 0.1 |
| P27b | ITS | Cutaneum cluster | CGGACAATTCTTGAACTCTGGT | (SEQ ID NO:30) | 0.2 |
| P28b | IGS | T. jirovecii | TGTGAGTCTATCGGGCGCTTG | (SEQ ID NO:31) | 0.2 |
| P29 | ITS | T. lignicola | TGTCCTCTGGAGTAATAAGTT | (SEQ ID NO:32) | 0.1 |
| P30 | ITS | Porosum cluster | TGAACGTCTAGTTATTATAACAA | (SEQ ID NO:33) | 0.2 |
| P31 | D1/D2 | T. gamsii | ATAAAGGTAATAGGAAGGTGG | (SEQ ID NO:34) | 0.2 |
| P32 | D1/D2 | T. laibachii/T. multisporum/ | CAGTCGTGTTTATTGGATTCA | (SEQ ID NO:35) | 0.2 |
| | | T. dulcitum/T. gracile | | | |
| P33 | ITS | Gracile cluster | TGAACGTCATTAGATCATAA | (SEQ ID NO:36) | 0.1 |
| P34b | IGS | Cryptococcus curvatus | GGTTTAAGATTGTATTGACTG | (SEQ ID NO:37) | 0.2 |
| P35 | IGS | T. dulcitum | AACGTACAAGTCCGGACATGA | (SEQ ID NO:38) | 0.2 |
| P36 | IGS | T. dermatis | ACGGTAGTTTGGAGTGAAGTGC | (SEQ ID NO:39) | 0.2 |
| P37 | IGS | T. asteroides/T. japonicum | GAGCAGCGAGCGACTTGGCAG | (SEQ ID NO:40) | 0.2 |
| P38 | IGS | T. asahii | TTCTCGACTGATGGCCTTGGT | (SEQ ID NO:41) | 0.2 |
| P39 | IGS | T. montevideense | GGGTCCTAATAGATGCCATGT | (SEQ ID NO:42) | 0.2 |
| P41b | IGS | T. coremiiforme | CACAGAGGTGCAAGAGGTGGG | (SEQ ID NO:43) | 0.2 |
| P42 | IGS | T. lactis | GCTTCGGAGACTTGGGTTTGC | (SEQ ID NO:44) | 0.4 |
| P43 | ITS | T. inkin/T. ovoides | ACTGTTCTACCACTTGACGCA | (SEQ ID NO:45) | 0.4 |
| P44b | D1/D2 | T. vadense | TTATCACATGCACTGGGGGAG | (SEQ ID NO:46) | 0.4 |
| P46 | IGS | T. domesticum | GCATGAGTGACGGCAGAGGTG | (SEQ ID NO:47) | 0.4 |
| P47 | IGS | T. laibachii | GTGCACAAGAAACATACTAAC | (SEQ ID NO:48) | 0.2 |

The invention also includes probes whose sequences are complementary to those presented in Table 2. The capture probes themselves may comprise, consist essentially of, or consist of these oligonucleotides. Fragments of the listed probes and complementary probes are also expected to be useful, as well as corresponding RNA probes.

Although the capture probes of the invention may be used in solution, they are particularly useful when bound to solid supports. In a preferred embodiment, the capture probes will be labeled with a detectable label, for example, a radioactive or fluorescent label. In one particularly preferred embodiment, the probes are bound to fluorescent beads to allow separation and identification of bound products. The capture probes may also be bound to a solid support, such as a multiwelled plate or a solid matrix to form a microarray. Solid phases or solid supports include, but are not limited to, those made of plastics, resins, polysaccharides, silica or silica-based materials, functionalized glass, modified silicon, carbon, metals, inorganic glasses, membranes, nylon, natural fibers such as silk, wool and cotton, and polymers, as will be know to those of skill in the art.

Examples of useful arrays include an array of color-coded beads (Luminex; Austin, Tex.), an array of radiofrequency-tagged beads (PharmaSeq; Monmouth Junction, N.J.), an array of nanocrystal encoded beads (Quantum Dot, Hayward, Calif.) or an array of radioisotopically labeled beads. A three dimensional microarray, as used herein, is any solid phase having three dimensions, wherein each microarray comprises a plurality of different biological molecules, preferably nucleic acid primers, attached to the surface. Thus, the location of each probe on the solid phase microarray enables the identification of each target species that is bound.

It is also an object of the invention to provide a method for detecting fungal pathogens using the capture probes of the invention. In one embodiment, the method comprises the steps of obtaining a set of beads with specific spectral addresses covalently bound to species-specific capture probes; contacting said fluorescent beads with a biological sample that may contain species for which said capture probes are specific under conditions such that the target species will bind to the capture probes; using a first laser to classify the target species/probes complexes by their spectral addresses; the target species may further be quantitated using fluorescent detection. Useful variations of this method will be apparent to those of skill in the art. In a particularly preferred embodiment, the capture probe is specific for at least one species of the genus Trichosporon. Examples of suitable capture probes are shown in Table 2. Complements of these probes and equivalent or corresponding RNA sequences will also be useful. By “complement” is meant any nucleic acid that is completely complementary over the entire length of the sequence, as understood in the art.

The sequences/probes of the invention may be used singly, but also may be advantageously used in combination with other sequences/probes of the invention, for example in combinations of 2, 3, 4, 5, 6, 7, 8, 9, 10, etc., up to an including all of the probes described herein.

A preliminary description of this invention has been published (4a).

### BRIEF DESCRIPTION OF THE DRAWINGS

FIG. 1. Phylogenetic tree (Maximum Likelihood—PAUP 4.0b10) of the D1/D2 LSU rDNA depicting probe numbers developed for Trichosporon species. [symbol] represents species not included in cluster probes.

FIG. 2. Results of Trichosporon brassicae probe (P1) tested with other species of Trichosporon. Nucleotide variations between the most closely related species are:

| | | |
|---|---|---|
| ATAGCCTAGTATCACATACAC | (SEQ ID NO:1) | Trichosponon brassicae CBS 6382 |
| ATAGCCTAtTATCACATACAC | (SEQ ID NO:49) | CBS 2481, CBS 4828, CBS 8641 |
| ATAGCCTAGgtTCACATACAC | (SEQ ID NO:2) | CBS 6721 |
| ATAGCCTAtTgTCACATACAC | (SEQ ID NO:50) | CBS 2478 |
| ATAGCCTAtTATtgCATACAC | (SEQ ID NO:51) | CBS 2466 |

FIG. 3. Results of T. scarabeorum probe tested with other species of Trichosporon and Cryptococcus sp. Sequences with off centered nucleotide variations i.e. T. dulcitum (CBS 8257) can yield false positive results. Samples were run in duplicates and the background fluorescence was subtracted.

| | | |
|---|---|---|
| CAGTAGGAATGTGGCTTCTTCGGA | (SEQ ID NO:52) | T. scarabeorum (CBS 5601) |
| tAtTAGGAATGTGGCTTCTTCGGA | (SEQ ID NO:53) | CBS 8257 |
| CAGTAGGAATGTaGCTTCTcCGGA | (SEQ ID NO:54) | CBS 2482 |
| CAtTAGGAATGTGGCTcCcTCGGg | (SEQ ID NO:55) | CBS 7743 |

FIG. 4. Effect of temperature on the specificity and signal intensity of T. ovoides probe (P13). T ovoides is represented by CBS 7556. Nucleotide variations between the most closely related species are

| | | |
|---|---|---|
| GTTTCACTGGTTCCATTGTGT | (SEQ ID NO:15) | T. ovoides |
| GTTTCACTGGgTCCATTGTGT | (SEQ ID NO:56) | CBS 5973, CBS 2479, CBS 4828, CBS 2481 |
| GTTTCACTGGgTCCATgGTGT | (SEQ ID NO:57) | CBS 5585 |

Other strains differed by 8 to 9 bp.

FIG. 5. Effect of various amount of capture probe on the signal performance of T. faecale (CBS 4828); T. guehoe (CBS 8521); T. inkin (CBS 5585)/T. ovoides (CBS7556); T. mucoides (CBS 7625); T. caseorum (CBS 9052) and T. sporotrichoides (CBS 8246). Samples were run in duplicates and the background fluorescence of each set of beads was subtracted.

FIG. 6. Effect of capture probe modification on the fluorescence signal of T. porosum (CBS 2040) and T. smithiae (CBS 8370). Probe 21 represents the 21 mer oligo: ATGAATCATGTTTATTGGACT (SEQ ID NO:58), whereas Probe 21b, represents the modified version represented by CATGAATCATGTTTATTGGACTC (SEQ ID NO:23). The sequence of probe 23: TGGATTTGAGTGATGGCAGTT (SEQ ID NO:25) was modified to TGGATTTGAGTGATGGCAGTT (SEQ ID NO:25) (P23b) by adding one bp at the 5′ and 3′ end.

FIG. 7. Effect of amplicon sizes on the fluorescence intensities of P11b: T. mucoides (CBS 7625); P18b: T. aquatile (CBS 5973); P16b: T. japonicum (CBS 8641); P25c: T. sporotrichoides (CBS 8246); P28b T. jirovecil (CBS 6864), P36: T. dermatis (CBS 2043) and P38: T. asahii (CBS 2749).

FIG. 8. Comparison of hybridization signals of P11 and P43 using 1-plex, 5-plex and 15-plex assay format. Each set of probes was tested individually and in a bead mix consisting of one, five and sixteen probes.

FIG. 9. Detection limits of genomic DNA using various quantities of genomic DNA. The DNA template in the PCR reaction ranged from 1 pg to 10 ng. After amplification, 5 μl of the PCR product was tested with its complementary probe sequence. The hybridization assay was carried at 55° C.

FIG. 10. Detection of amplified targets. Amplicons targeting its complementary probe sequence were serially diluted and tested using the described hybridization assay format. Strains and probes tested: P12: T. cutaneum (CBS 2466); P11: T. mucoides (CBS 7625); P13: T. ovoides (CBS 7556) and P36 T. dermatis (CBS 2043).

FIG. 11. Concentration curves of synthetic oligonucleotides using the standard hybridization assay format. Synthetic oligonucleotide targets for P2, P4 and P5 were reverse and complement of the capture probe sequences and were labeled with biotin at the 5′ end. R² represents logarithmic regression values.

### DETAILED DESCRIPTION OF THE INVENTION

### Materials and Methods

Strains and DNA Isolation

The examined strains (Table 1) were obtained from Centraaalbureau voor Schimmelcultures (CBS), Utrecht, The Netherlands; Portuguese Yeast Culture Collection (PYCC) and American Type Culture Collection (ATCC).

TABLE 1
List of strains studied.

| | |
|---|---|
| Trichosporon aquatile | CBS 5973 T |
| Trichosporon asahii | CBS 2479 T |
| Trichosporon asahii | CBS 8640 |
| Trichosporon asteroids | CBS 2481 T |
| Trichosporon brassicae | CBS 6382 T |
| Trichosporon caseorum | CBS 9052 T |
| Trichosporon cutaneum | CBS 2466 T |
| Trichosporon coremiiforme | CBS 2482 T |
| Trichosporon coremiiforme | CBS 2478 |
| Trichosporon dehoogii | CBS 8686 T |
| Trichosporon debeurmannianum | CBS 1896 T |
| Trichosporon dermatis | CBS 2043 T |
| Trichosporon dermatis | CBS 8381 |
| Trichosporon domesticum | CBS 8280 T |
| Trichosporon domesticum | CBS 8111 |
| Trichosporon dulcitum | CBS 8257 T |
| Trichosporon faecale | CBS 4828 T |
| Trichosporon gamsii | CBS 8245 T |
| Trichosporon gracile | CBS 8189 T |
| Trichosporon gracile | CBS 8518 |
| Trichosporon gracile | CBS 8519 |
| Trichosporon guehoae | CBS 8521 T |
| Trichosporon inkin | CBS 5585 T |
| Trichosporon japonicum | CBS 8641 T |
| Trichosporon jirovecii | CBS 6864 T |
| Trichosporon laibachii | CBS 5790 T |
| Trichosporon loubierii | CBS 7065 T |
| Trichosporon moniliiforme | ATCC46490 |
| Trichosporon montevideense | CBS 6721 T |
| Trichosporon mucoides | CBS 7625 T |
| Trichosporon multisporum | CBS 2495 T |
| Trichosporon ovoides | CBS 7556 T |
| Trichosporon porosum | CBS 2040 T |
| Trichosporon scarabeorum | CBS 5601 T |
| Trichosporon smithiae | CBS 8370 T |
| Trichosporon sporotrichoides | CBS 8246 T |
| Trichosporon vadense | CBS 8901 T |
| Trichosporon veenhuisii | CBS 7136 T |
| Cryptococcus curvatus | CBS 570 T |
| Cryptococcus magnus | CBS 140 T |
| Crypbococcus sp. | CBS 7743 |
| Cryptococcus sp. | PYCC4964 |
| Cryptococcuswrightensis | CBS 3131 T |
| Hyalodendron lignicola | CBS 222.34 T |
| Tsuchiyea wingfieldii | CBS 7118 T |

CBS: Centraalbureau voor Schimmelcultures;
ATCC: American Type Culture Collection;
PYCC: Portuguese Yeast Culture Collection.

DNA isolation was obtained from cell cultures grown overnight and employed the use of QIAmp Tissue kit (QIAGEN Inc) and a lysing enzyme derived from Trichoderma harzianum (Sigma Inc). This extraction method is described by Fell et al. (8).

PCR Conditions

DNA amplification was carried out with DNA extracted from pure cure cultures using three sets of primers targeting the ribosomal DNA regions (rDNA): a) large sub unit D1/D2 (LrDNA) region b) internal transcribed spacer regions (ITS) c) intergenic spacer region (IGS). The D1/D2 amplicons, which yielded amplicon sizes of ˜630 bp, were generated using the universal forward primer F63 (5′-GCATATCAATAAGCGGAGGAAAAG-3′) (SEQ ID NO:59) and the universal reverse primer, R635 (5′-GGT CCG TGT TTC AAG ACG-3′) (SEQ ID NO:60). The ITS regions (530 bp) were amplified using the forward primer ITS1 (5′TCCGTAGGTGAACCTGCG 3′) (SEQ ID NO:61) and the reverse primer ITS4 (5′TCCTCCGCTTATTGATATGC-3′) (SEQ ID NO:62). For IGS amplifications, three different sets of amplicons were generated using the reverse primer 5Srs (5′-AGCTTGACTTCGCAGATCGG-3′) (SEQ ID NO:63) with the forward primers: a) Lr12:5′-CTGAACGCCTCTAAGTC-AGAA-3′ (650-875 bp) (SEQ ID NO:64); b) Lr11:5′TTACCACAGGGATAACTGGC-3′ (950-1200bp) (SEQ ID NO:65) or c) IG1:5′CAGACGACTTGAATGGGAACG-3′(490-600bp) (SEQ ID NO:66). All PCR reverse primers were biotinylated at the 5′ end.

The reactions were carried out in microtubes containing Qiagen HotStarTaq Master Mix (QIAGEN Inc) with a final volume of 50 μl. The master mix contained: 10 ng to 1 pg of genomic DNA, 1×PCR buffer containing 1.5M MgCl₂, 200 μM of each dNTPs, 0.4 μM of forward and reverse primer pairs and 2.5 U of HotStarTaq DNA polymerase. The PCR reaction was performed for 35 cycles in a MJ Research PTC 100 thermocycler. The PCR program involved 15 min of initial activation step at 95° C., 30 sec of denaturating step at 95° C., 30 sec annealing at 50° C. and 30 sec extension at 72° C., followed by a 7 min final extension at 72° C. Samples were kept at 4° C. until further analysis. An agarose gel electrophoresis was performed to confirm the synthesis of amplicons.

Probe Development and Probe Coupling

Probe design at species level was based on sequence data from D1/D2, ITS 1&2 and IGS regions (28, 33). Probe selection was facilitated using visual sequence alignment employing Megalign Program (DNAStar). Areas displaying sequence divergence among the species were analyzed for probe selection. All probes were designed to be uniform in length (21 mer), however to avoid potential secondary structures or unstable delta G, some probe lengths were modified, resulting in probe sequences of 20 to 24 bp. The quality of the probe was assessed using the software program Oligo™ (Molecular Biology Insights Inc.).

The specificity of the prospective sequence was analyzed with a yeast database developed in our laboratory using the Mac Vector Program and GeneBank BLAST. The database sequences are accessible in GeneBank. Further probe validation was achieved by testing the performance of the probe on a captured probe hybridization format. Typically the probes were tested in a multiplex format of 5. The capture probes, which were complementary in sequence to the biotinylated strand of the target amplicon, were synthesized with a 5′ end Amino C12 modification (IDT—Coralville, Iowa). Each probe was covalently coupled to a different set of 5.6 μm polystyrene carboxylated microspheres using a standard carbodiimide method as described by Fulton et al. (11). Each microsphere set, (Miraibio-CA) contains unique spectral addresses by combining different concentrations of red and infrared fluorochromes. A typical reaction involved the coupling of 5×10⁶ microspheres resuspended in 25 μl of 0.1 M MES, pH 4.5 with a determined amount of probe (0.1 to 0.4 nmol). After successive vortexing and sonication steps, the beads were incubated twice with a final concentration of 0.5 μg/μl of EDC in the dark for 30 min at room temperature. The microspheres were washed with 1 ml of 0.02% Tween 20, followed by 1 ml 0.1% SDS. The beads were resuspended in 100 μl of TE buffer (10 mM Tris-HCl 1 mM EDTA, pH 8) and kept in the dark at 4° C.

Capture Probe Hybridization Assay

This assay is based upon detection of 5′biotin labeled PCR amplicons hybridized to specific capture probes covalently bound to the carboxylate surface of the microspheres. The 50 μl reaction mix, which was carried out in 96 well plates in the presence of a 3M TMAC (tetramethyl ammonium chloride/50 mM Tris, pH 8.0/1 mM EDTA, pH 8.0/0.1% SDS) solution, consisted of 5 μl of biotinylated amplicon diluted in 12 μl of 1×TE buffer (pH 8) and 33 μl of 1.5×TMAC solution containing a bead mixture of approx. 5000 microspheres of each set of probes. Prior to hybridization, the reaction mixture was incubated for 5 min at 95° C. with a PTC-100 Thermocycler (MJ Research). This step was followed by 15 min incubation at 55° C. After hybridization, the microspheres were pellet by centrifugation at 2250 rpm for 3 min with Eppendorf 5804 centrifuge. Once the supernatant was carefully removed, the plate was further incubated for 5 min at 55° C. and the hybridized amplicons were labeled for 5 min at 55° C. with 300 ng of the fluorescent reporter molecule, streptavidin R-phycoerythrin. Reactions were then analyzed on the Luminex 100. One hundred microspheres of each set were analyzed, which represents 100 replicate measurements. Each assay was run twice and the samples were run in duplicates. A blank and a set of positive and negative controls were included in the assay.

To test the detection limits of Luminex technology, several assays were conducted with various quantities (100 to 5 fmol) of biotinylated synthetic oligonucleotides targets, bearing the reverse and complement of the probe sequence. In addition, the sensitivity of the assay was conducted using serial dilutions of genomic DNA (10 to 1×10⁻³ ng) and amplicons (500 to 1×10⁻³ ng).

To test the multiplex capability of the assay, each individual set of D1/D2, ITS, and IGS probes were pooled together into a bead mix and tested in various multiplex formats. The multiplex array of D1/D2, ITS and IGS probes consisted of 18, 16 and 14 plex assays, respectively. In addition, probes were tested in 1 and 5 plex formats. Each plex assay was tested with amplicons derived from single species.

### Results

Trichosporon as a Test Model

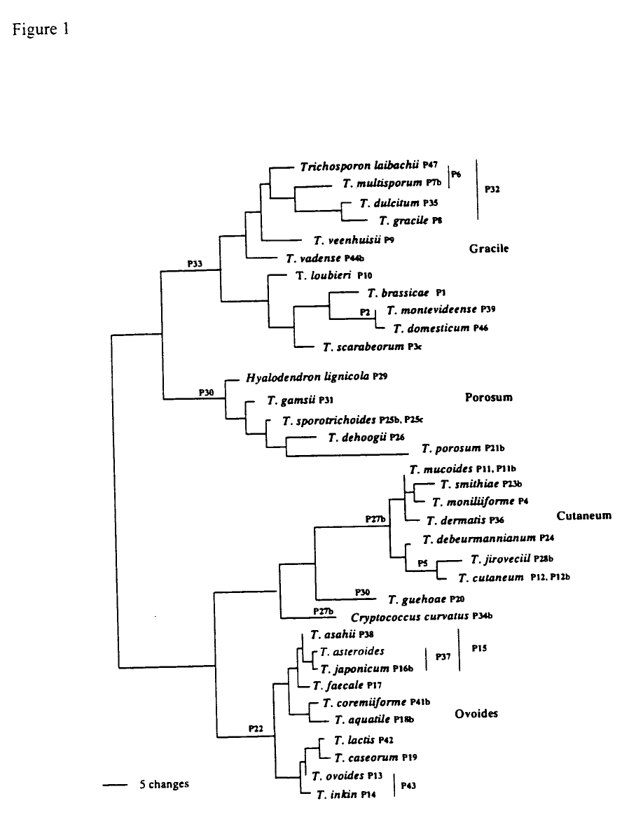

The genus Trichosporon was selected as our proof of concept model as this group comprises a large number of closely related species, some of which are virulent pathogens. A list of all the tested strains is represented in Table 1. This list includes all 33 species of the basidiomycetous yeast, Trichosporon (23). Two other strains belonging to the genus Cryptococcus (CBS 570: Cryptococcus curvatus) and Hyalodendron (CBS 222: Hyalodendron lignicola) were included based on the phylogenetic positions they occupy within Cutaneum and Porosum clades (FIG. 1).

Based on sequence analysis of the D1/D2 and ITS region, phylogenetic analyses were derived using PAUP*4 (maximum likelihood, random step-wise addition). The phylogenetic delineation obtained from the D1/D2 analysis segregates the species into four distinct clades: Gracile, Cutaneum, Porosum and Ovoides (FIG. 1). Of the four clades, Cutaneum and Ovoides contain most of the medically relevant species. For example T. mucoides T. cutaneum, T. asahii, T. asteroides, T. ovoides and T. inkin are the most commonly encountered pathogens (18). The other species are prevalent soil fungi, which are potentially opportunistic pathogens.

Probe Development

Species-specific probes and cluster specific probes were developed from sequence analysis of D1/D2, ITS and IGS regions. Initial experiments were designed to validate and determine the probe specificity and the stringency conditions required to discriminate among closely related species. A description of the species specific and cluster probe sequences and the rRNA region chosen for probe design is illustrated in Table 2. The probes were designed to have GC % content higher than 30%, Tm's higher than 50° C., and a length of 21 oligonucleotides. Some probes did not follow the described requirements. For example: T. vadense (P44), T. debeurmannianum (P24) and Porosum cluster (P30) probes displayed GC % content of 18%, 29% and 24%, respectively, whereas probes targeting T. gracile (P8), T. debeurmannianum (P24), Porosum cluster (P30), T. caseorum (P19) and C. curvatus (P34b) exhibited Tm values ranging from 45-49° C. In order to increase the hybridization efficiency, some probes underwent length modification by adding or subtracting 1-3 base pairs at the 5′ and/or 3′ end ie: T. porosum (P21b). Probes that seem to form hairpins or strong secondary structures and positive ΔG free energy of reaction were avoided. Also, those displaying runs of more than three G's or C's at the 5′ or 3′ end were not chosen. The location of the mismatches in the target sites was centered within the probe to avoid any potential cross reactivity.

FIG. 1 depicts 48 probes that were designed and tested in the present study. Individual species specific probes are in bold characters. Group specific probes to identify the four major lineages within the Trichosporon genus were designed in the ITS since this region exhibited less variability than D1/D2 region (27). All other species specific, or mini cluster probes were designed in one of the three rRNA regions (Table 2). The close phylogenetic relationship within the Ovoides clade, which contains four out of the six most relevant pathogenic species, facilitated the search for a group specific probe (P22) that can identify all the strains within the cluster. The inclusion of all members within the individual Cutaneum, Gracile and Porosum clades represented a challenge. For example, T. porosum and T. debeurmannianum are species not targeted by Porosum (P30) and Cutaneum cluster probes (P27b), respectively. Similarly, the Gracile cluster probe (P33) excludes the species T. montevideense and T. domesticum. In view of this limitation, specific probes were designed to target all species excluded from cluster probes. The exclusion of the species within their clades was due to inherent sequence divergence within the probe designing region or because the species diverged significantly from the cluster group. The latter applies to T. montevideense and T. domesticum, which showed sequence divergence, ranging from 2.5 to 5.7%, from members of the Gracile lade. The ITS tree topology depicted both species as a sister clade of the Gracile group.

Special attention was given to the medically important yeasts where duplicate probes were designed for selected species to confirm the presence or absence of the species in question. For the detection of T. mucoides, and T. cutaneum, which are commonly encountered pathogens, two species specific probes were designed in different regions of the rDNA (T. mucoides: P11(ITS) and P11b (IGS); T. cutaneum: P12 (ITS) and P12 b (IGS). Other species, such as T. inkin, T. ovoides, T. cutaneum, and T. asahii were targeted by species specific and cluster probes (Table 2). Probes for T. loubieri (P10) and T. dermatis (P36), which are new opportunistic pathogens within the genus, were also identified by species specific probes. Identification of T. asteroides, an agent implicated in superficial infections, relied on a process of elimination due to difficulty finding an adequate probe sequence. Thus, two probes with broader specificities were designed to target T. asteroides. Probe 15, which includes the species: T. asteroides/T. japonicum/T. asahii and P37, comprising T. asteroides and T. japonicum. With the inclusion of an additional probe, P16 b (T. japonicum), we were able to resolve the species T. japonicum from T. asteroides.

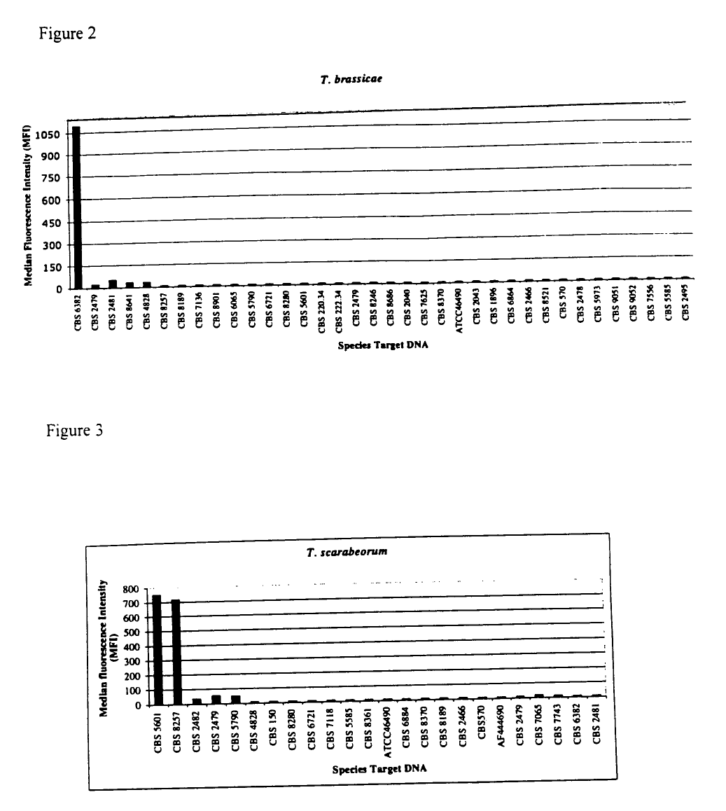

Each species-specific probe was tested against the complementary target amplicon: positive controls (perfect match), negative controls (more than three mismatches) and cross-reactive groups (one to three mismatches). The Luminex assay format, which was employed to test the specificities of the probes, includes members of Trichosporon and other fungal genera that can potentially cross-react with the probe sequence. Results on 21 mer length probes demonstrated that the selected hybridization assay conditions discriminate probe sequences differing by one or two base pairs depending on the position of the mismatch, which influence the extent of the hybrid destabilization. An example of probe specificity is illustrated in FIG. 2. The D1/D2 probe, T. brassicae (P1), was tested against a battery of DNA (Trichosporon spp) that displayed several mismatches from the selected probe sequence. The results illustrated that mismatches located in the center or at positions 9-10-11 from the 5′ or 3′ end were discriminated using the assay conditions. However, when the mismatches were located near the 3 or 5′ end, the assay lost specificity. To illustrate the extent of potential cross-reactivity, T. scarabeorum probe sequence was designed to differ by two bp at the 5′ end from T. dulcitum. The results demonstrated that the location of the mismatches are critical and can lead to false positive results if mismatches are not centered (FIG. 3). In view of the high cross reactivity, this probe was not included in Table 2, and another sequence (P3c) was selected to target T. scarabeorum (Table 2). As expected, the median fluorescent intensity of perfect matches was higher than the probe target hybrids displaying mismatches.

Hybridization Assay Optimization Conditions

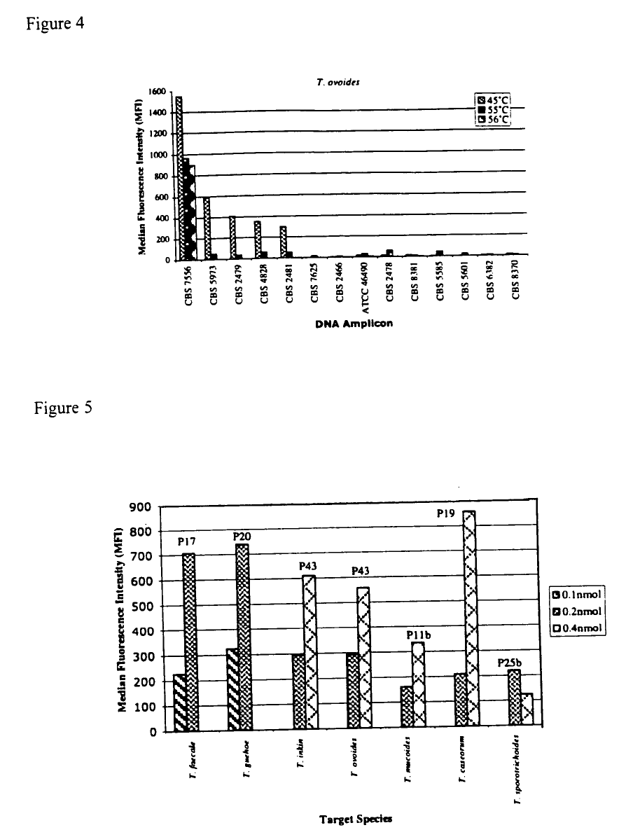

The assay conditions involved the use of TMAC, which is a quaternary ammonium salt agent that increases the stringency conditions allowing discrimination among oligonucleotides differing by one bp. Under 3M TMAC, the hybridization conditions were dependent on the oligonucleotide length and not upon the base composition. The hybridization conditions were optimized by adjusting hybridization temperatures to provide adequate sensitivity and stringency conditions necessary for detection of the target species. FIG. 4 shows the performance of T. ovoides (P13) at different hybridization temperatures ranging from 45 C to 56 C. Hybridization at 45 C did not provide the discrimination between perfectly matched and mismatched sequences since other species, eg. T. aquatile (CBS 5973), T. asahii (CBS 2479), T. faecale (CBS 4828) and T. asteroides (CBS 2481) cross-reacted with the probe. A point to note is that the fluorescent intensity of these species, which differed by only one bp, were lower than the perfect matched species, T. ovoides (CBS 7556). In contrast, hybridization at 55° C. provided a good signal and stringent conditions needed to discriminate between these closely related species (FIG. 4). Further optimization of assay conditions was achieved by comparing the hybridization efficiency against various amounts of capture probes conjugated to the microspheres. These coupling amounts were optimized for each probe. For example, T. guehoe (P20) and T. faecale (P17) probes showed a 75% and 61% increase in signal performance at 0.2 nmol as compared to 0.1 nmol of probe (FIG. 5). Others, like the group specific ITS T. inkin/T. ovoides (P43) probe and the D1/D2 T. caseorum (P19) probes improved their signals by 51.8% and 75% when the capture probe coupling amount was increased from 0.2 to 0.4 mnol, respectively (FIG. 5). An increase in the amount of capture probe did not always correlate with higher signals. For instance, probes like T. sporotrichoides (P25b) showed a 43.5% decrease in signal when the coupling probe amount was increased from 0.2 nmol to 0.4 nmol (FIG. 5). This “hook effect” occurs when the microspheres are over-conjugated (Luminex, personal communication). In this scenario, the numbers of conjugation sites became limited by high amounts of the capture probe. This is within normal experimental expectation.

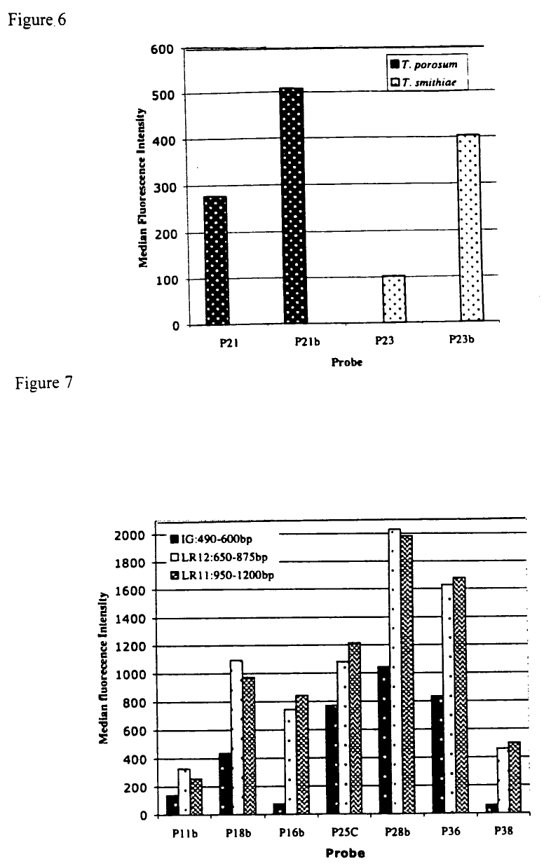

Probes that did not perform satisfactorily after testing for optimal probe coupling amount, underwent sequence or length modification by adding bases at the 3′or 5′ end. For instance, a 74.5% and 45% increase in signal was observed when a total of two base pairs, located at the 3′ end and 5′ end were added to the probe sequences of T. smithiae (P23b), and T. porosum (P21b), respectively (FIG. 6). Addition of bases can enhance the hybridization efficiency by increasing the amount of hybridized material. However, in some instances, probe lengthening led to a decrease in probe specificity. For example, when Porosum cluster probe (Table 2) was modified by adding 2 bp: ATGAACGTCTAGTTATTATAACAA (SEQ ID NO:67) (bold character denotes addition of bp), the probe lost specificity and cross-reacted with T. smithiae (P23b).

Different probes exhibited different signal intensities, ranging from ˜200 to 2000 above background levels. This wide range in hybridization signals can be attributed to different hybridization and coupling efficiencies of captures probes, variations in the efficiency of fluorescence labeling and to different association/dissociation constants of the probe sequences Also, the surrounding nucleotide composition of the probe annealing area is known to have an impact on the strength of the signal.

The signal-to-background ratio (S:B) for all tested probes fluctuated between ˜3.3 to ˜61.8. The highest signal to background ratio was observed for T. jirovecil (P28b) with a S:B of ˜61.8, followed by T. montevideense/T. domesticum (P2) with a S:B of ˜59. In contrast, the ITS probe designed to target the species, T. sporotrichoides (P25b) exhibited a S:B of ˜3.30. In view of the poor signal to background ratio of P25b, another probe sequence (P25c) was chosen to avoid ambiguous identifications. This new probe (P25c), which was designed in the IGS region, exhibited a S:B of ˜28. Overall, our signal to background ratios were adequate and positive results correspond to normalized MFI values, which are twice the background levels.

Amplicon Size

Amplicon sequences under 300 bp are usually recommended in multiple hybridization assay formats as they allow probe sequences to successfully compete with the complementary strand of the amplicon. As a result, the reaction occurs in a fast and efficient manner. However, our studies demonstrate that efficient hybridization reactions can occur with amplicons longer than 600 bp. Using three different sets of primers (IGS1/5sR, Lr12/5sR, Lr11/5sR), we examined the effect of amplicon sizes on the hybridization signal of the species: T. mucoides (P11b), T. aquatile (P18b), T. jirovecil (P28b), T. japonicum (P16b), T. dermatis (P36), T. sporotrichoides (P25c), and T. asahii (P38) (FIG. 7). The primer set, IGS1/5sR was used to generate the shortest segments ranging in length from 490 to 600 bp, whereas Lr12/5sR yielded 650 to 875 bp amplicon fragments. For longer target amplicons (950 to 1,200 bp), the primer combination Lr11/5sR was employed. The wide range of species length polymorphisms, with each set of primers, is attributed to indel and repeat areas, which are common characteristics of the IGS region (5, 34). Surprisingly, lower hybridization signals were documented with shorter target amplicons (490 to 600 bp) generated with IGS1/5sR (FIG. 7). In contrast, significantly higher signals were observed with amplicon fragments over >600 bp, which were obtained with the primer pairs Lr11/5sR or Lr12/5sR (FIG. 7). A similar trend was observed for the DI/D2 probes, T. gracile (P8) and T. veenhuisii (P9). Both probes exhibited nearly 38% increase in signal with target amplicons of approx. ˜1200 bp (ITS 1/R635) as opposed to ˜600bp fragments obtained with the primer set F63-R635 (data not shown). Not all probes performed better with longer fragments. For instance, an ITS probe designed to target the species T. laibachii/T. multisporum (P6) failed to produce hybridization signal when tested against a target sequence of 1,200 bp. However, a positive signal was obtained when a shorter amplicon of ˜600 bp was used (data not shown). Secondary structures generated with McFold Program (data not shown), showed that the secondary structure area where the complementary probe sequence is located (pos 360-380) is similar for both amplicon fragments, where a section of the sequence appears to flank an internal and a multibranch loop. However, the overall secondary structure of the large fragment has a more complex pattern, consisting of many hairpin loops, stem structures and a main multibranch with 17 hairpin loops. Most probably, these secondary structures can induce bending and distortion, which can affect the probe binding efficiency.

Multiplex Reactions

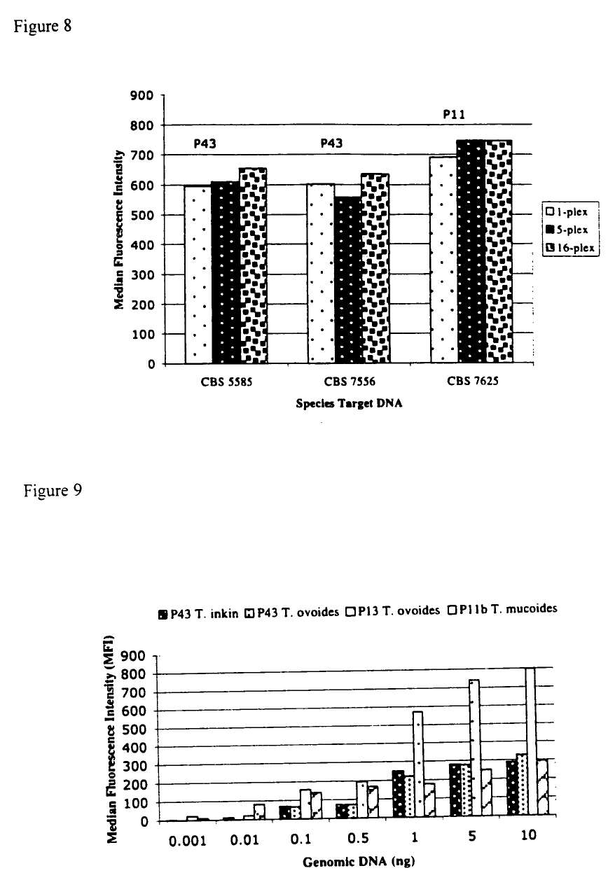

To test the multiplex capability of Luminex technology, different sets of probes were pooled together and tested using a single target PCR per well. Fluorescence signal intensity for D1/D2, ITS and IGS probes tested in multiplex formats were found to be similar to those observed in uniplex (non multiplexed format) or quintuplex format. For example, FIG. 8, illustrates the performance of P11 and P43 when tested in different multiplex formats consisting of 1-plex (1 probe), 5-plex (5 probes) and 15 plex (15 probes). The signal intensities of probes tested in various plex formats were not significantly different, suggesting the potential capability of this technology to test simultaneously different sets of probes without compromising the fluorescent signal. However, P32 and P35 were the only probes displaying a significant reduction in fluorescence intensity of up to 50% when tested in a 16 and 18plex format, respectively (data not shown).

Genomic and Amplicon Detection Limits

Clinically relevant fungal species were employed to test the sensitivity of the assay using serial dilutions of genomic DNA, ranging from 10 ng to 1×10⁻³ ng. DNA quantification was determined with NanoDrop® ND-1000 spectrophotometer using an absorbance of 260 nm. Reactions were performed in duplicate and the experiment run twice. P43 (T. inkin/T. ovoides), P13 (T. ovoides) and P11b (T. mucoides) gave robust signals when the amount of genomic DNA ranged from 10 ng to 1 ng. But lower signals were recorded when genomic DNA ranged between 500 pg to 100 pg (FIG. 9). Similar results were obtained with P12 (T. cutaneum), P14 (T. inkin) and P38 (T. asahii) (data not shown). Beyond 10 pg the probe signals were barely detectable, except for T. mucoides (P11b), which displayed a MFI˜80. The results demonstrate that the present method is able to detect as low as 10 to 100 pg of genomic DNA with MFI signals ranging from 67 to 150 above background levels. An exception was P12 (T. cutaneum), exhibiting a detection limit of 500 pg (data not shown). However, better detection limits were obtained when using higher amounts of PCR product in the assay format. For instance, detection limits as low as 10 pg were obtained for P43 (T. inkin/T. ovoides), P11b (T. mucoides), P38 (T. asahii) and P11 (T. mucoides) when the amount of PCR product was increased to 15 μl (data not showed).

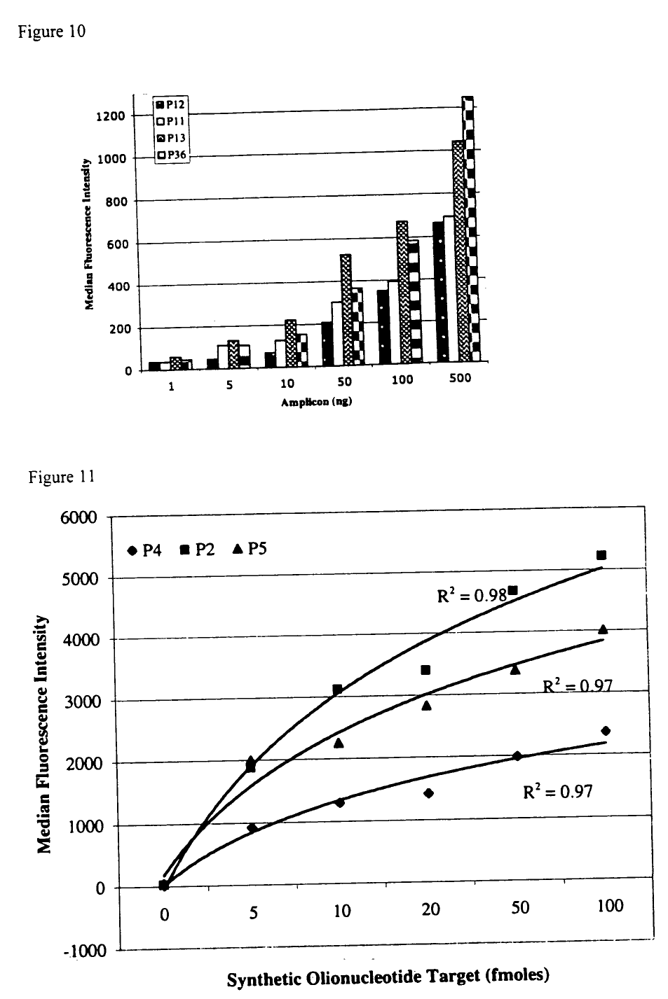

To determine the detection limits of the amplification products, amplicons were serially diluted from 500 ng to 1×10⁻³ ng. Prior to quantification, PCR products were purified with Qiagen Quick-spin (QIAGEN Inc). As shown in FIG. 10, there is a steady increase in signal as the amount of amplification product is increased from 1 ng to 500 ng. Below 1 ng, no increase in signal was documented and the MFI values were close to background levels (data not shown). Based on the presented data, this study shows that PCR sensitivity for the tested probes ranges between 1 to 5 ng, with signal intensities over 50 MFI, except for P12, which showed <50 MFI values at 1 ng.

### Discussion

Herein, we describe and test a reliable molecular technique, which combines PCR, hybridization kinetics and flow cytometry to target group specific and species specific strains of the medically important genus Trichosporon . A total of 48 probes were designed and tested using a hybridization assay format combined with Luminex 100 technology. This technology provides a rapid means of species detection with the flexibility to allow the detection of species in a multiplex format. The present hybridization assay format combined with Luminex technology, provided sufficient specificity and discrimination to differentiate closely related species. A probe hierarchical approach was followed to target species specific probes and group specific probes encompassing closely related species within a clade. The combined use of several species specific probes and general probes can alleviate ambiguities and provides further information related to the phylogenetic placement of the species. This approach can be of extreme value in clinical settings, where redundancy in results is needed to ascertain an accurate diagnosis.

As in any hybridization assay with capture probes, optimization of assay parameters was needed to facilitate stable duplex formations with high specificity. The use of 3M TMAC, in combination of 55° C. hybridization temperature, provided the conditions necessary to achieve the high stringency conditions to discriminate between sequences differing by only one bp. TMAC, which is known to equalize AT and CG by base pair stability, has been incorporated in hybridization assay formats because it allows different sets of probes with different characteristics to be used under identical hybridization conditions (17, 21). The equalization of the melting points of different probes with a 3M or 4M solution of TMAC has enhanced the duplex yields (22).

To achieve probe specificity, it was of paramount importance to locate any mismatch in the center of the probe sequence, otherwise the assay led to false positive results. Mismatches in the center are known to have a more profound effect on the equilibrium state than mismatches near the 5′ or 3′ end (13). A study based on the kinetic effects of mismatches located at the first, fifth and seventh base pair in a 13 mer oligonucleotide, showed that the variation in Ka (association rate constant) is the highest when the location is at midpoint from 5′ or 3′ end (13).

Other factors such as probe length and attachment efficiency can have significant effects on the specificity and performance of some probes. For instance, the addition of two bp to the probe sequences of T. smithiae (P23b) and T. porosum (P21b) improved their hybridization efficiency. Probe lengthening has been reported to enhance hybridization efficiency by increasing the amount of hybridized material (31) and also can have significant impacts on probe equilibrium states by increasing the enthalpy and entropy of the probe-target duplex reaction. The impact on the equilibrium state is dependent upon the base pair composition addition and the sequence context (nearest neighbor effect) (27, 36). However, adding few bp to some probe sequences does not always improve probe performance, as was the case of Porosum lade specific probe (P30b). Similar effects have been reported by others, where a substantial decrease in resolution and specificity was found when probes underwent few nucleotides length modifications (3). Reports in the literature, indicate that when a length of a probe is increased, a mismatched base pair in the probe-target duplex will have a marginal effect on the stability of the duplex. In this scenario, the effect of free energy penalty associated from mismatches basepairs, become a fraction of the total free energy binding (2). This would explain why mismatches associated in shorter sequences promote higher levels of destabilization in a duplex (3, 19).

Overall, factors related to probe design and sequence content was found of uttermost importance for the success of this methodology. For instance, a sequence displaying a string of six repeats as portrayed in T. vadense probe (AGATCATAACATAAAAAAACTT) (SEQ ID NO:68) was found to be nonspecific. Similarly, the location of the probe appeared to have an effect in the probe performance. For example, sequences selected near 100 bp from the 5′ end performed poorly or did not yield any signal. On the contrary, sequences selected from the middle or close to the 3′ end of the alignment tend to perform better. Apparently, binding site areas closer to the 3′ end allows better interaction between the capture probe and the 5′ end biotinylated amplicon by minimizing potential formation of secondary structures near the duplex formation site.

The observed wide range of fluorescence signals upon hybridization of different probes might be associated with nucleotide sequence, duplex stability and secondary structures. Some of these factors are: a) base stacking interactions associated with probe sequences. For example, unpaired bases stacking on the end of a duplex, as is the case when the target overlaps the capture probe, may affect the duplex yield (39), b) presence of internal hairpin or secondary structure in the probe. Although we avoided probes with hairpin structures, few internal complementary bases within the sequence of the capture probe might lead to minor secondary structures affecting the formation and duplex yield, c) presence of secondary structure conformations near the probe-target binding area. DNA target can easily fold back upon itself to form helixes and even more complicated structures as a result of the Watson Crick base pairing. These structural conformations, if close to the binding area, might prevent or partially interfere with duplex formation, d) different association/dissociation constants, which have an immediate effect on the kinetic parameters of probe-target interaction.

The sensitivity of the assay, as determined by P43 (T. inkin/T. ovoides), P38 (T. asahii), P11 and P11b (T. mucoides) demonstrated that this method enabled the detection of 10 pg of genomic DNA template in the PCR reaction, except for P13 (T. ovoides) and P14 (T. inkin), which required 100 pg of genomic DNA (FIG. 9). Assuming that the genome size of Trichosporum spp. is similar to Cryptococus neoformans (24MB) and the average molecular weight of a dsDNA base pair is 660 Dalton, 10 pg of genomic DNA correspond to a detection limit of ˜380 genome molecules. Detection limits ranging from 1,659 to 189,753 genome molecules has been reported by others using Luminex technology for the identification of bacterial pathogens (6).

After correcting for PCR product length and assuming there are 200 rRNA gene copy numbers in Trichosporon sp, the PCR product limit of detection for P36 (T. dermatis), P13 (T. ovoides) and P11 (T. mucoides) ranged from 20.2 to 25.2 fmol. This represents a detection limit of 6.08×10⁷ copies for T. dermatis, and 7.55×10⁷ copies for T. mucoides and T. ovoides. In contrast, P14 (T. inkin) and P43 (T. inkin/T. ovoides) required 50.4 fmol (1.51×10⁸ copies). Other probes, particularly, P38 (T. asahii) and P12 (T. cutaneum), displayed detection limits of 189 fmol (5.68×10⁸ copies) and 252 fmol (7.58×10⁸ copies), respectively. These detection limits represent cutoff values above background signals, where the signal is ˜2 times above background levels, once the background has been subtracted. Our calculated limit of detection could be more sensitive than the above values of further optimized. Relatively higher PCR detection limits, ranging from 0.25 to 0.1 fmol or 10⁶ to 10⁷ amplicon copies were reported by Dunbar et al. (2003), who used 20 mer synthetic oligonucleotide targets to determine the sensitivity of the PCR product in the hybridization assay. In contrast, we employed >600 bp PCR fragments. We speculate the difference in detection limits is attributed to different hybridization kinetics and efficiencies when longer amplicons are employed. For instance, when we tested synthetic oligonucleotide targets, a much higher sensitivity was observed with signals ˜1000 MFI at 5 fmol levels (FIG. 11).

In summary, the methods described in the present application can be executed in clinical settings for the identification of Trichosporon species. This medically important fungal pathogen was used as our proof of concept model for the development of a comprehensive assay aimed at the identification of all the medically relevant fungal species. This assay uses Luminex technology, which has the potential capability to provide multiplex analysis combined with a high-throughput system. This non-washed captured probe hybridization assay involves few and simple steps that can be performed in less than 50 min after amplification products are generated. The specificity and sensitivity of the assay allowed discrimination of 1 bp among the species and allowing the detection of 10² to 10⁴ genome copies in the PCR reaction. Limits of detection in the hybridization reaction ranged from 10⁷ to 10⁸ amplicon target copies. In addition to the multiplexing capability, were as many as 100 different species can be analyzed in a single well, the ease of use, accuracy and low cost of operation are few of the conveniences of this technology. In addition, this bead based assay allows the creation of different clinical testing platforms by combining different set of microspheres. Any modification to the modules will simply involve the mixing of the proper set of microspheres. In contrast, density microarray methods are less flexible since they require the printing of new plates with specialized equipment.

### REFERENCES

References cited herein are hereby incorporated by reference and are listed below for convenience:
1. Abd-Elsalam, K. A., N. Ibrahim, M. A. Abdel-Satar, M. S. Khalil, and J. A. Verreett. 2003. PCR identification of Fusarium genus based on nuclear ribosomal-DNA sequence data. Afr. J. Biotech. 2:82-85.
2. Abou-ela, F., D. Koh, I. Tinoco Jr., and F. J. Martin. 1985. Base-base mistmatches. Thermodynamics of double helix formation for dCA3XA3G+dCT3YT3G (X, Y=A,C,G,T) Nucleic Acids. Res. 13:3944-3948.
3. Armstrong B, M. Stewart, and A. Mazumder. 2000. Suspension arrays for high throughput multiplexed single nucleotide polymorphism genotyping. Cytometry 40:102-108.
4. Carlson D., J. Y. Lo, D. Ally, and E. Ubil. 2000. Microsphere assay for Y chromosome SNPs. Proceedings of Eleventh International Symposium on Human Identification, October 10-13 Mississippi.
4a. Diaz M. R. and J. W. Fell. 2004. High-Throughput Detection of Pathogenic Yeasts of the Genus Trichosporon . J. Clin. Microbiol. 42:3696-3706.
5. Diaz M. R., J. W. Fell, T. Boekhout, and B. Theelen. 2000. Molecular sequence analyses of the intergenic spacer (IGS) associated with rDNA of the two varieties of the pathogenic yeast, Cryptococcus neoformans. Syst and Appl. Microbiol: 23:535-545.
6. Dunbar, S. A., C. A. Vander Zee, K. G. Oliver, K. L. Karem, and J. W. Jacobson. 2003. Quantitative, multiplexed detection of bacterial pathogens: DNA and protein applications of the Luminex LabMap system. J. Microbiol. Meth. 53:245-252.
7. Fell, J. W. 1995. rDNA targeted oligonucleotide primers for the identification of pathogenic yeasts in a polymerase chain reaction. J. Ind. Microbiol. 14:475-477.
8. Fell, J. W., T. Boekhout, A. Fonseca, G. Scorzetti, and A. Statzell-Tallman. 2000. Biodiversity and Systematics of basidiomycetous yeasts as determined by large subunit rDNA D1/D2 domain sequence analysis. Int. J. Syst. Bact. 50:1351-1371.
9. Fleming, R. V., T. J. Walsh, and E. J. Anaissie. 2002. Emerging and less common fungal pathogens. Infect. Dis. Clin. North. Am. 16:915-933.
10. Fujita, S. I, Y. Senda, S. Nakaguchi, and T. Hashimoto. 2001. Multiplex PCR using internal transcribed spacer 1 and 2 regions for rapid detection and identification of yeast strains. J. Clin. Microbiol. 39:3617-3622.
11. Fulton R, R McDade, P. Smith, L. Kienker, and J. Kettman. 1997. Advanced multiplexed analysis with the FlowMetrix system. Clin. Chem 43:1749-1756.
12. Gardes, M., T. J. White, J. A. Fortin, T. D. Bruns, and J. W. Taylor. 1991. Identification of indigenous and introduced symbiotic fungi in ectomycorrhizae by amplification of nuclear and mitochondrial ribosomal DNA. Can. J. Bot. 69:180-190.
13. Gotoh, M., Y. Hasegawa, Y. Shinohara, M. Schimizu, and M. Tosu. 1995. A new approach to determine the effect of mistmaches on kinetic parameters in DNA hybridization using an optical biosensor. DNA Res. 2: 285-293.
14. Haynes, K. A, T. J. Westerneng, J. W. Fell, and W. Moens. 1995. Rapid detection of pathogenic fungi by polymerase chain reaction amplification of large subunit ribosomal DNA. J. Med. Vet. Mycology. 33:319-325.
15. Itoh, T., U. Hosokawa, N. Kondera, N. Toyazaki, and Y. Asada. 1996. Disseminated infection with Trichosporon asahii. Mycoses. 39:195-199.
16. Kanj S. S., K. Welty-Wolf, J. Madden, V. Tapson, M. A. Baz, and D. Davis. 1996. Fungal infections in lung and heart-lung transplant recipients, report of 9 cases and review of the literature. Medicine: 75:142-156.
17. Kwon-Chung, K. J. and J. E. Bennett. 1992. In: Medical Mycology. Lea and Febiger pp 866, Pennsylvania, USA.
18. Kiesling, T., M. R. Diaz, A. Statzell-Tallman, and J. W. Fell. 2002. Field identification of marine yeasts using DNA hybridization macroarrays, p. 69-80. In: Hyde, KD, ST Moss and LLP Vrijmoed (eds), Fungi in Marine Environments, Fungal Diversity Press, Hong Kong.
19. Livshits M. A., and A. D. Mirzabekov. 1996. Theoretical analysis of the kinetics of DNA hybridization with gel-immobilized oligonucleotides. Biophys. J. 71: 2795-801.
20. Makimura, K., S. Murayama, and H. Yamaguchi. 1994. Detection of a wide range of medically important fungi by polymerase chain reaction. J. Med. Microbiol. 40:358-364.
21. Maskos, U. and E. M. Southern. 1993. A study of oligonucleotide reassociation using large arrays of oligonucleotides synthesized on a large support. Nucleic Acids Res. 21:4663-4669.
22. Maskos, U. and E. M. Southern. 1992. Parallel analysis of oligodeoxyribonucleotide (oligonucleotide) interactions. I. Analysis of factors influencing duplex formation. Nucleic Acids Res. 20:1675-1678.
23. Middelhoven, W. J., G. Scorzetti, and J. W. Fell. 2003. Systematics of the anamorphic basidiomycetous yeast genus Trichosporon Behrend with the description of five novel species. Int. J. Sys. Evol. Microbiol. In press.
24. Nagai H., Y. Yamakami, A. Hashimoto, I. Tokimatsu, and M. Nasu. 1999. PCR detection of DNA specific for Trichosporon species in serum patients with disseminated trichosporonosis. J. Clin. Microbiol. 37:694-699.
25. Oliver, K., L. Patel, J. Kemp, J. Daves, L. Bell and, R. Zivin. 1999. The Luminex LabMAP system: a rapid, homogeneous, multianalyte platform. Society for Biomolecular Screening Meeting, Edinbugh, UK September 1999.
26. Padhye, A. A., S. Verghese, P. ravichandran, G. Balamurugan, L. Hall, P. Padma, and M C. Fernandez. 2003. Trichosporon loubieri infection in a patient with adult polycystic kidney disease. J. Clin. Microbiol. 41:479-482.
27. Santa Lucia, J. Jr. 1998. A unified view of polymer, dumbbell and oligonucleotide DNA nearest-neighbor thermodynamics. Proc. Natl. Acad. Sci. 95:8602-8606.
28. Scorzetti, G., J. W. Fell, A. Fonseca and A. Statzell-Tallman. 2002. Systematics of basidiomycetous yeasts: a comparison of large subunit D1D2 and internal transcribed spacer rDNA regions. FEMS Yeast Res. 2:495-517.
29. Spiro, A., M. Lowe, and D. Brown. 2000. A bead-based method for the multiplexed quantitation of DNA sequences using flow cytometry. Appl. Environ. Microbiol. 66:4258-4265.
30. Steel, A. B., T. M. Herne, and M. J. Tarlov. 1998. Electrochemical quantitation of DNA immobilized on gold. Anal. Chem. 70:4670-4677.
31. Staib, F. 1987. Cryptococcosis in AIDS-mycological, diagnostic and epidemiological observations. AIDS-Forsch 2:363-382.
32. Sugita, T. A., M. Nikishikawa, and T. Shinoda. 1998. Rapid detection of species opportunistic yeast Trichosporon by PCR. J. Clin. Microbiol. 36:1458-1460.
33. Sugita, T. A., M. Nikishikawa, R. Ikeda, and T. Shinoda. 1999. Identification of medically relevant Trichoporon species based on sequences of internal transcribed spacer regions and construction of a database for Trichosporon identification. J. Clin. Microbiol. 37:1985-1993.
34. Sugita, T. A, M. Nakajima, R. Ikeda, T. Matsuhima, and T. Shinoda. 2002. Sequence analysis of ribosomal DNA intergenic spacer 1 regions of Trichoporon species. J. Clin. Microbiol. 40:1826-1830.
35. Sutton, D. A. 2002. Laboratory evaluation of new antifungal agents against rare and refractory mycoses. Curr. Opin. Infect. Dis. 15: 576-582.
36. Tsourkas, A., M. A. Behlke, S. D. Rose, and G. Bao. 2003. Hybridization kinetics and thermodynamics of molecular beacons. Nucleic Acids Res. 31:1319-1330.
37. Walling D. M., D. J. McGraw, W. G. Merz, J. E. Karp, and G. M. Hutchins. 1987. Disseminated infection with Trichosporon beigelii. Rev. Infect. Dis. 9:1013-1019.
38. Walsh, T. J. 1989. Trichosporonosis. Infect. Dis. Clin. North Am. 3:43-52.
39. Williams, J. C., S. C. Case-Green, S. C. Mir., and E. M. Southern. 1994. Studies of oligonucleotide interactions by hybridization to arrays: the influence of dangling ends on duplex yield. Nucleic. Acids Res. 22:1365-1367.
40. Ye, F., M. S. LI, J. D. Taylor, Q. Nguyen, H. M. Colton, W. M. Casey, M. Wagner, M. P. Weiner, and J. Chen. 2001. Fluorescent microsphere-based readout technology for multiplexed human single nucleotide polymorphism analysis and bacterial identification. Hum. Mutat. 17:305-316.

We claim:

1. An isolated nucleic acid sequence comprising a DNA sequence selected from Table 2, a complement thereof, or a corresponding RNA sequence.

2. A capture probe comprising a nucleic acid sequence of claim 1.

3. A composition comprising a capture probe of claim 2 that is bound to a solid support.

4. The composition of claim 3 wherein the solid support is a fluorescent bead.

5. A composition containing a plurality of capture probes as claimed in one of claims 2-4.

6. The composition of claim 5 comprising at least 5 of said capture probes.

7. A method for detecting a fungal pathogen comprising the steps of providing at least one capture probe of claim 2;
contacting said capture probe(s) with a biological sample that may contain target species of nucleic acid for which said capture probe(s) are specific under conditions such that the target species will become bound to the probe to produce a hybridized product;
detecting the presence or absence of hybridized product, the presence of said hybridized product being indicative of the presence of said fungal pathogen.

8. The method of claim 7 that further comprises quantitating the hybridized product.

9. The method of claim 7 wherein the capture probe is bound to a solid support.

10. A method for detecting fungal pathogens comprising the steps of obtaining a set of fluorescent beads covalently bound to capture probes;
contacting said fluorescent beads with a biological sample that may contain amplicons of target species for which said capture probes are specific under conditions such that said amplicons will become bound to the probe to produce a hybridized product;
using a first laser to classify the beads by their spectral addresses; and
detecting the presence or absence of said hybridized product, the presence of said hybridized product being indicative of the presence of said fungal pathogen.
quantitating hybridized biotinylated amplicons using fluorescent detection.

11. The method of claim 10 wherein said first laser has a wavelength of 635 nm.

12. The method of claim 10 wherein the hybridized biotinylated amplicons are quantified with a 532 nm laser.

13. The method of claim 10, wherein the capture probe is specific for a species or strain from the genus Trichosporon.

14. The method of claim 10 wherein the capture probes are selected from Table 2.

15. A kit comprising at least one capture probe of claim 2, 3 or 4, optionally including instructions for use.

16. The kit of claim 15 containing a plurality of capture probes as claimed in one of claims 2-4.

17. The kit of claim 16 comprising at least 5 of said capture probes.
